(19) United States
(12) Patent Application Publication
(10) Pub. No.: US 2023/0277215 A1
(43) Pub. Date: Sep. 7, 2023

(54) IMPLANTABLE MEDICAL DEVICE WITH SHORT LINEAR ACTUATION DELIVERY MECHANISM

(71) Applicant: BOSTON SCIENTIFIC SCIMED, INC., Maple Grove, MN (US)

(72) Inventors: Joshua Mark Inouye, Brooklyn Park, MN (US); Ryan Robert Davis, Plymouth, MN (US)

(73) Assignee: BOSTON SCIENTIFIC SCIMED, INC., MAPLE GROVE, MN (US)

(21) Appl. No.: 18/115,911

(22) Filed: Mar. 1, 2023

Related U.S. Application Data
(60) Provisional application No. 63/315,612, filed on Mar. 2, 2022.

(51) Int. Cl. A61B 17/34; A61F 2/24

(52) U.S. Cl. CPC A61B 17/3468; A61F 2/2436; A61B 2017/1205

(57) ABSTRACT

An assembly for delivering an implantable medical device, the assembly includes an implantable medical device including an attachment feature and a delivery device that is adapted to releasably secure the implantable medical device, the delivery device including an attachment element adapted to create an interference fit with the attachment feature of the implantable medical device. The delivery device is adapted to enable a user to selectively disengage the interference fit between the attachment element of the delivery device and the attachment feature of the implantable medical device with a short linear motion when the delivery device is positioned proximate a delivery site for the implantable medical device.

### CROSS-REFERENCE TO RELATED APPLICATIONS

This application claims the benefit of priority of U.S. Provisional Application No. 63/315,612 filed Mar. 2, 2022, the entire disclosure of which is hereby incorporated by reference.

### TECHNICAL FIELD

The present disclosure pertains to medical devices, and methods for manufacturing and using medical devices. More particularly, the disclosure is directed to implantable medical devices having a short linear actuation delivery mechanism.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an assembly for delivering an implantable medical device includes an implantable medical device including an attachment feature and a delivery device that is adapted to releasably secure the implantable medical device, the delivery device including an attachment element adapted to create an interference fit with the attachment feature of the implantable medical device. The delivery device is adapted to enable a user to selectively disengage the interference fit between the attachment element of the delivery device and the attachment feature of the implantable medical device with a short linear motion when the delivery device is positioned proximate a delivery site for the implantable medical device.

Alternatively or additionally, the interference fit between the attachment element of the delivery device and the attachment feature of the implantable medical device may enable the implantable medical device to pivot relative to the delivery device before the interference fit is disengaged by the user.

Alternatively or additionally, the delivery device may include an attachment sheath and the attachment element may include a release cord adapted to extend through the attachment sheath and engage the attachment feature of the implantable medical device, the release cord including a distal region that is adapted to extend back into the attachment sheath.

Alternatively or additionally, the short linear motion may include moving the attachment sheath a distance of about ten millimeters or less in a proximal direction, thereby freeing the distal region of the release cord from the attachment sheath.

Alternatively or additionally, the release cord may be adapted to enable the release cord to be retracted proximally into the attachment sheath once the attachment sheath has been moved proximally.

Alternatively or additionally, the distal region may have an outer diameter that is greater than an outer diameter of a rest of the release cord.

Alternatively or additionally, the distal region may have an outer diameter that is equal to an outer diameter of a rest of the release cord.

Alternatively or additionally, the assembly may further include a moveable element having a first configuration in which the moveable element is adapted to hold the attachment element in the interference fit with the attachment feature and a second configuration in which the moveable element is adapted to release the attachment element from its interference fit with the attachment feature.

Alternatively or additionally, the one or more attachment features may include an attachment cavity formed within the implantable medical device, the one or more attachment elements may include an attachment member that fits within the attachment cavity, and the moveable element may be adapted to move within the attachment member between a release position in which the attachment member is released from an interference fit with the attachment cavity and a secure position in which the attachment member is held in an interference fit with the interference cavity.

Alternatively or additionally, the short linear motion may include moving the moveable element a distance of about ten millimeters or less in a proximal direction.

Alternatively or additionally, the moveable element may include a pin that is moveable within a lumen extending within the attachment member.

Alternatively or additionally, the one or more attachment features may include an attachment cavity formed within the implantable medical device, the attachment cavity including a toroidal spring, and the one or more attachment elements may include a rod having an annular groove adapted to accommodate the toroidal spring.

As another example, an assembly for delivering an implantable medical device includes an implantable medical device including an attachment feature and a delivery device adapted to releasably secure the implantable medical device. The delivery device includes an attachment sheath, the attachment sheath adapted to be linearly moveable a short distance. The delivery device includes a release cord adapted to extend through the attachment sheath and releasably engage the attachment feature, the release cord including a distal region that is adapted to extend back into the attachment sheath.

Alternatively or additionally, the attachment sheath may be adapted to be linearly moveable a short distance of about ten millimeters or less, thereby freeing the distal region of the release cord from the attachment sheath.

Alternatively or additionally, the release cord may be adapted to enable the release cord to be retracted proximally into the attachment sheath once the attachment sheath has been moved proximally.

Alternatively or additionally, the distal region may have an outer diameter that is greater than an outer diameter of a rest of the release cord.

Alternatively or additionally, the distal region may have an outer diameter that is equal to an outer diameter of a rest of the release cord.

As another example, an assembly for delivering an implantable medical device includes an implantable medical device having an attachment cavity formed within the implantable medical device. The assembly includes a delivery device adapted to releasably secure the implantable medical device. The delivery device includes an attachment member that is adapted to releasably fit within the attachment cavity, the attachment member including a lumen extending within the attachment member. The delivery device includes a moveable element slidingly disposed within the lumen, the moveable element moveable between a first configuration in which the moveable element is adapted to hold the attachment element in the interference fit with the attachment cavity and a second configuration in which the moveable element is adapted to release the attachment member from its interference fit with the attachment cavity.

Alternatively or additionally, the moveable element may be adapted to move axially within the lumen a distance of about ten millimeters or less in order to move between the first configuration and the second configuration.

Alternatively or additionally, the moveable object may include a pin.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A through 1E are schematic views which together show deployment of an implantable medical device using an illustrative delivery device;

FIG. 2A through 2E are schematic views which together show deployment of an implantable medical device using an illustrative delivery device;

FIG. 3A is a side view of an illustrative assembly including an illustrative implantable medical device and an illustrative delivery device;

FIGS. 3B and 3C are side views which together show deployment of the illustrative implantable medical device of FIG. 3A;

FIG. 4 is a schematic view of an illustrative assembly including an implantable medical device and a delivery device;

FIG. 5 is a schematic view of an illustrative assembly including an implantable medical device and a delivery device;

FIG. 6 is a schematic view of an illustrative assembly including an implantable medical device and a delivery device;

FIG. 7 is a schematic view of an illustrative assembly including an implantable medical device and a delivery device;

FIGS. 8A and 8B are schematic views which together show an illustrative assembly including an implantable medical device and a delivery device;

FIG. 9 is a perspective view of an illustrative adjustable endoluminal mitral valve ring implant; and

FIGS. 9A and 9B are schematic views which together show an illustrative assembly for delivering portions of the illustrative adjustable endoluminal mitral valve ring implant of FIG. 9.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

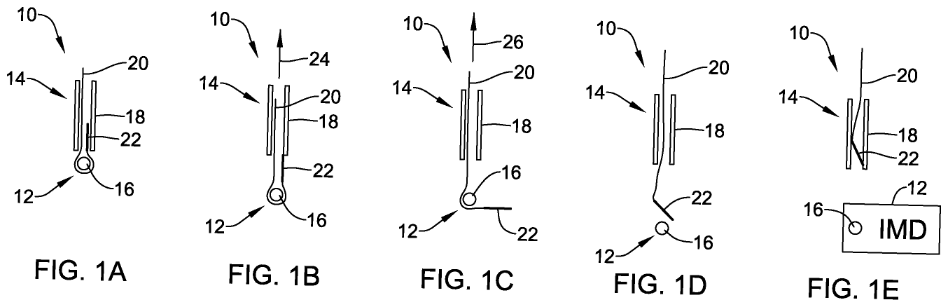

FIGS. 1A through 1E are schematic views which together show deployment of an implantable medical device using an illustrative delivery device. FIGS. 1A through 1E show an illustrative assembly 10 that includes an implantable medical device 12 and a delivery device 14. The implantable medical device 12 includes an attachment feature 16. For clarity, the attachment feature 16 is shown in FIGS. 1A through 1D by itself. The implantable medical device 12 is schematically shown in FIG. 1E. The attachment feature 16 may be any structure on or in the implantable medical device 12 that may be used for releasably securing the delivery device 14 to the implantable medical device 12. In some cases, the attachment feature 16 may simply be a rod or pin that has been added to the implantable medical device 12 or has been integrally formed as part of the implantable medical device 12. In some cases, the attachment feature 16 may include an aperture through which a release cord may be extended.

The implantable medical device 12 generically represents any of a variety of different implantable medical devices. In some cases, the implantable medical device 12 generically represents any of a variety of different implantable medical devices that can be delivered via a transcather delivery. One example of such an implantable medical device 12 is an LAAC (left atrial appendage closure) device. Another example of such an implantable medical device 12 is a cardiac implant such as a transcatheter valve implant or a valve repair device. Other implantable medical devices 12 are also contemplated.

The delivery device 14 is shown schematically, and only the portions of the delivery device 14 that are involved with releasably securing the implantable medical device 12 to the delivery device 14 are shown. The delivery device 14 includes an attachment sheath 18 that may be a metallic or polymeric cylinder, a coil or a hypotube, for example. The delivery device 14 also includes a release cord 20 that extends through the attachment sheath 18 in order to releasably engage the attachment feature 16 of the implantable medical device 12. The release cord 20 may be a thread, for example, with a heavier end on it. The release cord 20 may be a metallic wire, for example. In some cases, the release cord 20 includes a distal tip region 22 that has an outer diameter that is larger than an outer diameter of a rest of the release cord 20. The distal tip region 22 may be stiffer than a rest of the release cord 20, in order to make it more difficult for the distal tip region 22 to be removed from the attachment sheath 18.

FIG. 1A shows the implantable medical device 12 secured relative to the delivery device 14, with the release cord 20 extending out of the attachment sheath 18 and around the attachment feature 16 of the implantable medical device 12, with the distal tip region 22 tucked back up into the attachment sheath 18. As shown in FIG. 1A, the implantable medical device 12 is secured relative to the delivery device 14 but can articulate, i.e., pivot about an axis extending through the attachment feature 16.

Moving from FIG. 1A to FIG. 1B, it can be seen that the attachment sheath 18 has been moved a short distance in a direction indicated by an arrow 24. In use, this would generally be a movement in a proximal direction. In some cases, the attachment sheath 18 is moved in a linear direction for a distance that may be about ten millimeters or less. In some cases, the attachment sheath 18 may be moved in a linear direction for a distance that may be about five millimeters or less.

In some cases, a method of securing the implantable medical device 12 to the delivery device 14 may be seen in moving from FIG. 1B to FIG. 1A. As seen in FIG. 1B, the release cord 20 may be disposed about the attachment feature 16 with the distal tip region 22 adjacent but exterior to the attachment sheath 18. Moving the attachment sheath 18 a short distance in a direction opposite that indicated by the arrow 24 will cause the distal tip region 22 to become nested within an interior of the attachment sheath 18, thereby securing the attachment feature 16 (and hence the implantable medical device 12) relative to the delivery device 14.

While not shown, the attachment sheath 18 itself either extends proximally to a point at which a user can manipulate a relative position of the attachment sheath 18, or the delivery device 14 includes one or more structures that operably engage the attachment sheath 18 and extend proximally such that a use can engage the one or more structures in order to move the attachment sheath 18 proximally, for example.

As a result of having been moved a short distance in the direction indicated by the arrow 24, it can be seen in FIG. 1B that the distal tip region 22 is no longer constrained by being tucked into an interior of the attachment sheath 18. As a result, and as shown in FIG. 1C, the release cord 20 may be pulled in a direction indicated by an arrow 26 in order to retract the release cord 20 into the interior of the attachment sheath 18, without moving the attachment sheath 18 any further.

Progressing to FIGS. 1D and 1E, it can be seen that as the release cord 20 continues to be pulled in the direction indicated by the arrow 26, the distal tip region 22 of the release cord 20 is moved further away from the implantable medical device 12, and eventually may be pulled entirely into the interior of the attachment sheath 18. While pulling the distal tip region 22 of the release cord 20 completely into the interior of the attachment sheath 18 may not be necessary when deploying the implantable medical device 12, this can aid in actually deploying the implantable medical device 12 by moving the release cord 20 (and the distal tip region 22 thereof) more completely out of the way.

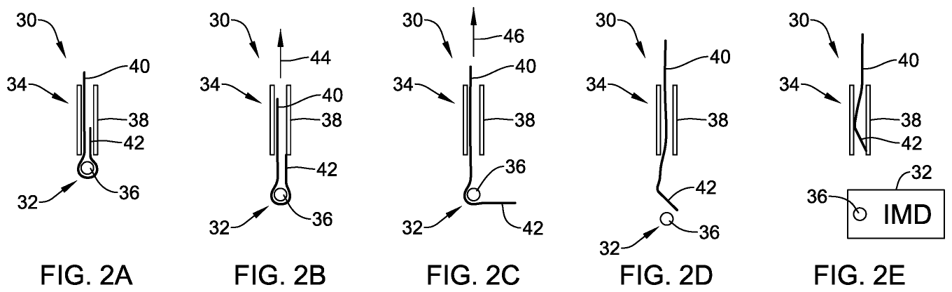

FIGS. 2A through 2E are schematic views which together show deployment of an implantable medical device using an illustrative delivery device. FIGS. 2A through 2E show an illustrative assembly 30 that includes an implantable medical device 32 and a delivery device 34. The implantable medical device 32 includes an attachment feature 36. For clarity, the attachment feature 36 is shown in FIGS. 2A through 2D by itself. The implantable medical device 32 is schematically shown in FIG. 2E. The attachment feature 36 may be any structure on or in the implantable medical device 32 that may be used for releasably securing the delivery device 34 to the implantable medical device 32. In some cases, the attachment feature 36 may simply be a rod or pin that has been added to the implantable medical device 32 or has been integrally formed as part of the implantable medical device 32. In some cases, the attachment feature 36 may include an aperture through which a release cord may be extended.

The delivery device 34 is shown schematically, and only the portions of the delivery device 34 that are involved with releasably securing the implantable medical device 32 to the delivery device 34 are shown. The delivery device 34 includes an attachment sheath 38 that may be a cylinder, a coil or a hypotube, for example. The delivery device 34 also includes a release cord 40 that extends through the attachment sheath 38 in order to releasably engage the attachment feature 36 of the implantable medical device 32. In some cases, the release cord 40 includes a distal tip region 42 that may get tucked into an interior of the attachment sheath 38. In some instances, the release cord 40 has a constant outer diameter, including the distal tip region 42.

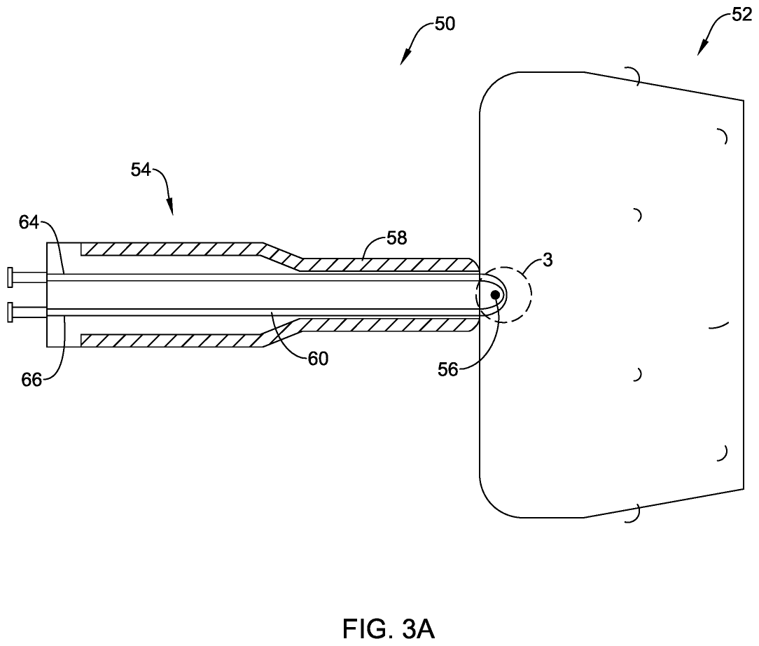

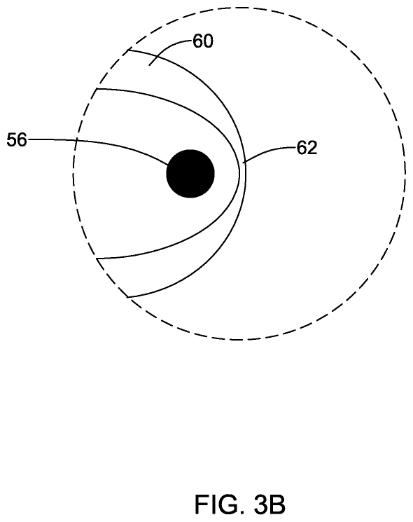

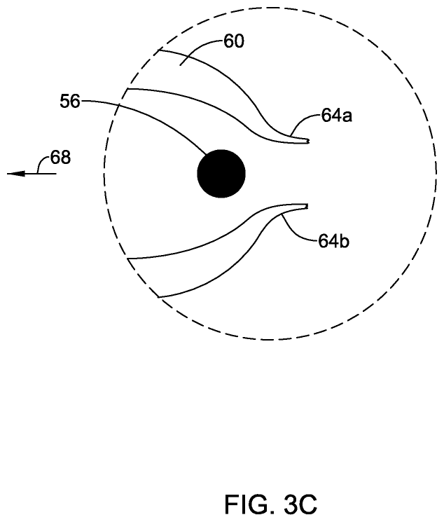

FIG. 2A shows the implantable medical device 32 secured relative to the delivery device 34, with the release cord 40 extending out of the attachment sheath 38 and around the attachment feature 36 of the implantable medical device 32, with the distal tip region 42 tucked back up into the attachment sheath 38. As shown in FIG. 3A, the implantable medical device 32 is secured relative to the delivery device 34 but can articulate, i.e., pivot about an axis extending through the attachment feature 36.

Moving from FIG. 2A to FIG. 2B, it can be seen that the attachment sheath 38 has been moved a short distance in a direction indicated by an arrow 44. In use, this would generally be a movement in a proximal direction. In some cases, the attachment sheath 38 is moved in a linear direction for a distance that may be about ten millimeters or less. In some cases, the attachment sheath 38 may be moved in a linear direction for a distance that may be about five millimeters or less.

In some cases, a method of securing the implantable medical device 32 to the delivery device 34 may be seen in moving from FIG. 2B to FIG. 2A. As seen in FIG. 2B, the release cord 40 may be disposed about the attachment feature 36 with the distal tip region 42 adjacent but exterior to the attachment sheath 38. Moving the attachment sheath 38 a short distance in a direction opposite that indicated by the arrow 44 will cause the distal tip region 42 to become nested within an interior of the attachment sheath 38, thereby securing the attachment feature 36 (and hence the implantable medical device 32) relative to the delivery device 34.

While not shown, the attachment sheath 38 itself either extends proximally to a point at which a user can manipulate a relative position of the attachment sheath 18, or the delivery device 34 includes one or more structures that operably engage the attachment sheath 38 and extend proximally such that a use can engage the one or more structures in order to move the attachment sheath 38 proximally, for example.

As a result of having been moved a short distance in the direction indicated by the arrow 44, it can be seen in FIG. 2B that the distal tip region 42 is no longer constrained by being tucked into an interior of the attachment sheath 38. As a result, and as shown in FIG. 2C, the release cord 40 may be pulled in a direction indicated by an arrow 46 in order to retract the release cord 40 into the interior of the attachment sheath 38, without moving the attachment sheath 38 any further.

Progressing to FIGS. 2D and 2E, it can be seen that as the release cord 40 continues to be pulled in the direction indicated by the arrow 46, the distal tip region 42 of the release cord 40 is moved further away from the implantable medical device 32, and eventually may be pulled entirely into the interior of the attachment sheath 38. While pulling the distal tip region 42 of the release cord 40 completely into the interior of the attachment sheath 38 may not be necessary when deploying the implantable medical device 32, this can aid in actually deploying the implantable medical device 32 by moving the release cord 40 (and the distal tip region 42 thereof) more completely out of the way.

FIG. 3A is a side view of an illustrative assembly 50. The illustrative assembly 50 includes an implantable medical device 52 and a delivery device 54. The implantable medical device 52 includes an attachment feature 56 that may be an integral part of the implantable medical device 52. In some cases, the attachment feature 56 may be separately manufactured and subsequently secured to the implantable medical device. In this particular instances, the implantable medical device 52 is an LAAC (left atrial appendage closure device). Additional details regarding the implantable medical device 52 may be found in US 2021/0015494, which publication is incorporated by reference herein.

The delivery device 54 includes an outer housing 58 and a tether 60 that extends through the outer housing 58 and engages the attachment feature 56 of the implantable medical device 52. As better seen in FIG. 3B, which is an enlarged view of a portion of FIG. 3A has a weakened portion 62 where the tether 60 extends around the attachment feature 56. As a result, and as shown for example in FIG. 3C, a sharp force applied to the tether 60, in particular, pulling on each of a first portion 64 of the tether 60 and a second portion 66 of the tether 60, will cause the tether 60 to break, forming a first broken end 64a and a second broken end 66a. Consequently, both portions 64 and 66 of the tether 60 may be withdrawn in a direction indicated by an arrow 68 from the attachment feature 56, thereby releasing the implantable medical device 52. Applying the sharp force to the tether 60 in order to break the tether 60 may be considered as being achieved through a short motion that may be ten millimeters or less, or perhaps even five millimeters or less.

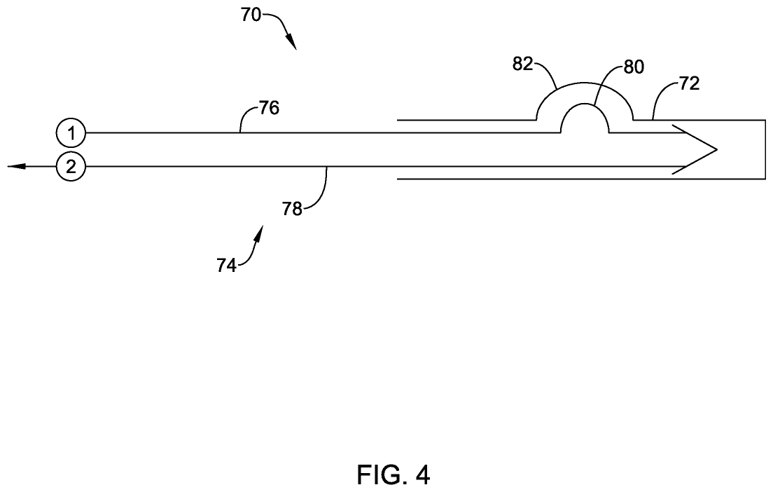

FIG. 4 is a schematic view of an illustrative assembly 70 including an implantable medical device 72 and a delivery device 74. The delivery device 74 includes a first wire 76 and a second wire 78. The first wire 76 forms a jog 80 that fits into a transverse cavity 82 that is formed within the implantable medical device 72. The first wire 76 forms an interference fit between the jog 80 and the transverse cavity 82 that secures the implantable medical device 72 to the delivery device 74. In some cases, the presence of the second wire 78, which remains static, prevents removal of the first wire 76. Consequently, removing the second wire 78 permits removal of the first wire 76, thereby releasing the interference fit between the jog 80 and the transverse cavity 82. Upon removal of the jog 80 from the transverse cavity 82, the implantable medical device 72 is released in place and the delivery device 74 may be withdrawn.

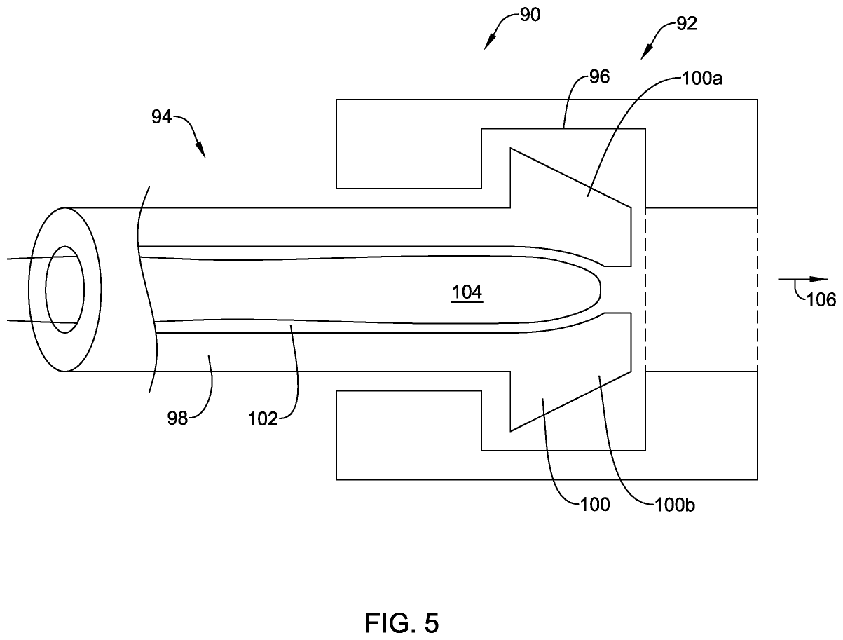

FIG. 5 is a schematic view of an illustrative assembly 90 including an implantable medical device 92 and a delivery device 94. The implantable medical device 92 includes an attachment cavity 96 that may be a closed cavity or an open cavity. The delivery device 94 includes an attachment member 98. The attachment member 98 includes a distal end 100 that has an enlarged profile, relative to the rest of the attachment member 98. The distal end 100 may be considered as forming an interference fit with the attachment cavity 96.

The attachment member 98 includes a lumen 102 that runs the length or at least substantially the length of the attachment member 98. The lumen 102 is adapted to accommodate a moveable element 104 that is slidingly disposed within the lumen 102. In some cases, the moveable element 104 is a pin, for example. When the moveable element 104 is extended within the lumen 102, as shown in FIG. 5, the moveable element 104 fills the lumen 102 and thus prevents the attachment member 98 from collapsing down onto itself, which would permit the distal end 100 of the attachment member 98 from forming an interference fit with the attachment cavity 96. Pushing the moveable element 104 further in a direction indicated by an arrow 106 will push components 100a and 100b of the distal end 100 further into the attachment cavity 96. Withdrawing the moveable element 104 in an opposing direction allows the components 100a and 100b of the distal end 100 to move towards each other, and thus out of engagement with the attachment cavity 96.

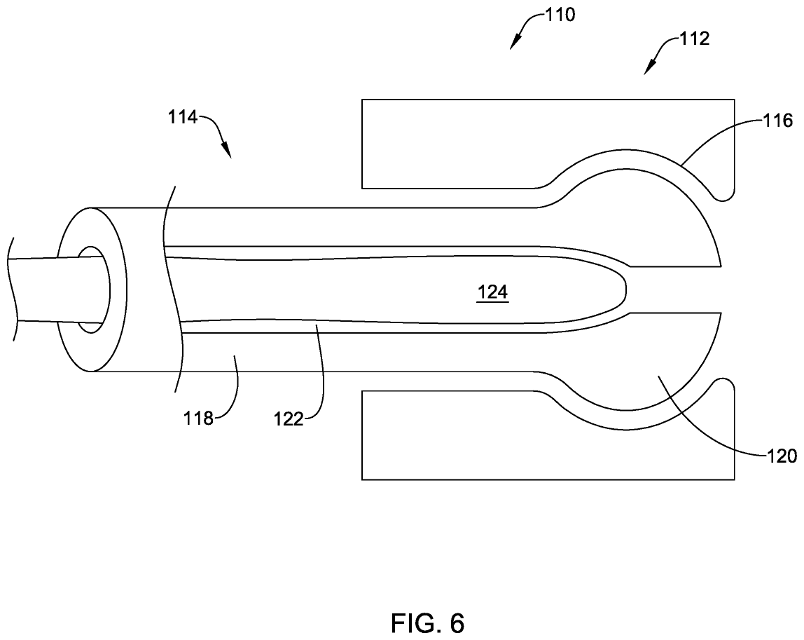

FIG. 6 is a schematic view of an illustrative assembly 110 including an implantable medical device 112 and a delivery device 114. The implantable medical device 112 includes an attachment cavity 116. The delivery device 114 includes an attachment member 118. The attachment member 118 includes a distal end 120 that has an enlarged profile, relative to the rest of the attachment member 118. The distal end 120 may be considered as forming an interference fit with the attachment cavity 116. In some cases, the distal end 120 may have a spherical or semispherical profile, for example.

The attachment member 118 includes a lumen 122 that runs the length or at least substantially the length of the attachment member 118. The lumen 122 is adapted to accommodate a moveable element 124 that is slidingly disposed within the lumen 102. In some cases, the moveable element 124 is a pin, for example. When the moveable element 124 is extended within the lumen 122, as shown in FIG. 6, the moveable element 124 fills the lumen 122 and thus prevents the attachment member 118 from collapsing down onto itself, which would permit the distal end 120 of the attachment member 118 from forming an interference fit with the attachment cavity 116.

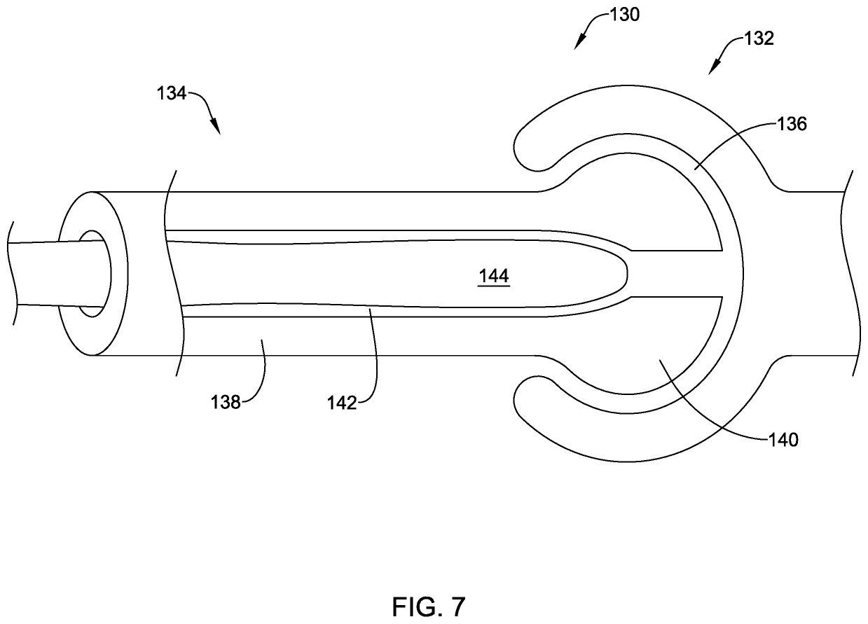

FIG. 7 is a schematic view of an illustrative assembly 130 including an implantable medical device 132 and a delivery device 134. The implantable medical device 132 includes an attachment cavity 136 that can be considered as being an open cavity that permits both rotation and articulation between the implantable medical device 132 and the delivery device 134. The delivery device 124 includes an attachment member 128. The attachment member 128 includes a distal end 140 that has an enlarged profile, relative to the rest of the attachment member 138. The distal end 140 may be considered as forming an interference fit with the attachment cavity 136. In some cases, the distal end 140 may have a spherical or semispherical profile, for example.

The attachment member 138 includes a lumen 142 that runs the length or at least substantially the length of the attachment member 138. The lumen 142 is adapted to accommodate a moveable element 144 that is slidingly disposed within the lumen 132. In some cases, the moveable element 144 is a pin, for example. When the moveable element 144 is extended within the lumen 142, as shown in FIG. 7, the moveable element 144 fills the lumen 142 and thus prevents the attachment member 138 from collapsing down onto itself, which would permit the distal end 140 of the attachment member 138 from forming an interference fit with the attachment cavity 136.

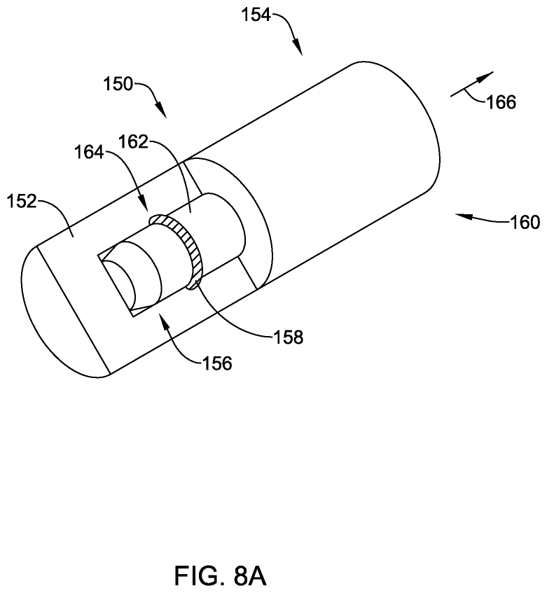

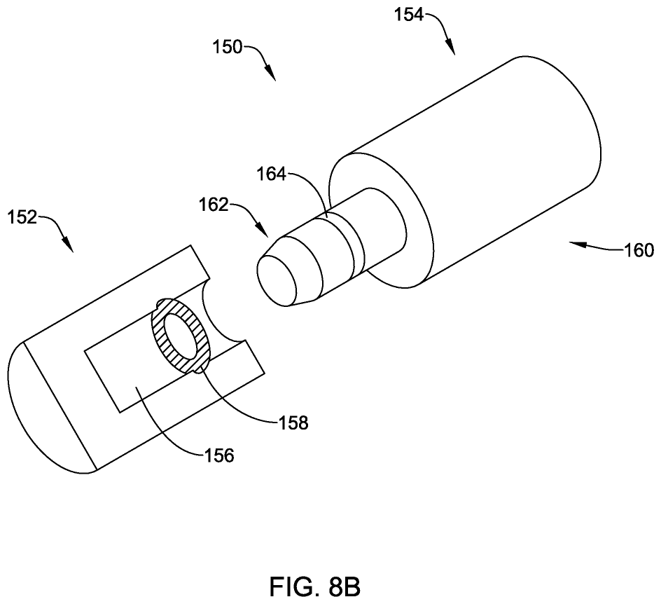

FIGS. 8A and 8B together are schematic perspective views of an illustrative assembly 150. The illustrative assembly 150 includes an implantable medical device 152 and a delivery device 154. The implantable medical device 152 includes an attachment cavity 156. It will be appreciated that the implantable medical device 152 is shown partially cutaway, in order to visualize the attachment cavity 156. In some cases, as shown, the implantable medical device 152 includes a toroidal spring 158.

The delivery device 154 includes an elongate member 160 having a reduced diameter portion 162 that is adapted to fit into the attachment cavity 156 of the implantable medical device 152. The reduced diameter portion 162 includes an annular groove 164 that is adapted to releasably engage the toroidal spring 158. When the elongate member 160 is advanced towards the implantable medical device 152 such that the reduced diameter portion 162 enters the attachment cavity 156 of the implantable medical device 152, the toroidal spring 158 engages the annular groove 164, thereby securing the implantable medical device 152 relative to the delivery device 154.

In order to release the implantable medical device 152 from the delivery device 154, the elongate member 160 may be pulled in a direction indicated by an arrow 166. The interference fit between the toroidal spring 158 and the annular groove 164 may be overcome by applying an appropriate force.

As shown, the toroidal spring 158 is mounted within the attachment cavity 156 (within the implantable medical device 152) and the annular groove 164 is formed within the reduced diameter portion 162 of the elongate member 160 (part of the delivery device 154). In some cases, the toroidal spring 158 may instead be secured relative to the reduced diameter portion 162 of the elongate member 160, and the annular groove 164 may be disposed within the attachment cavity 156.

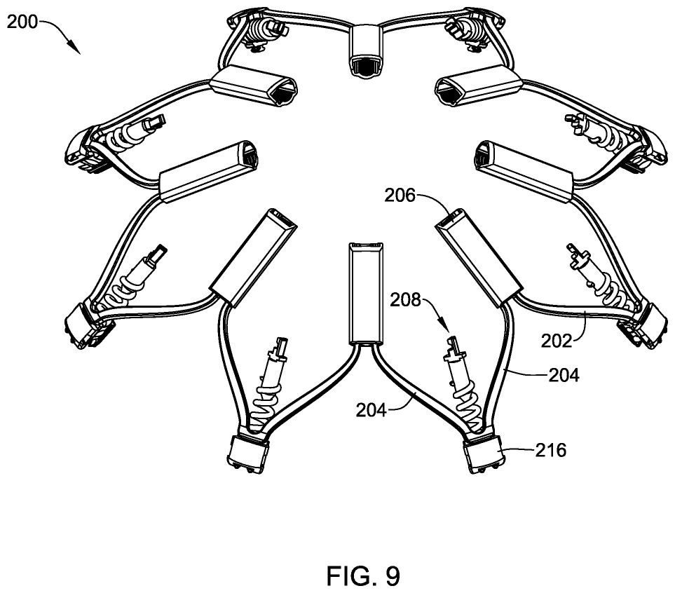

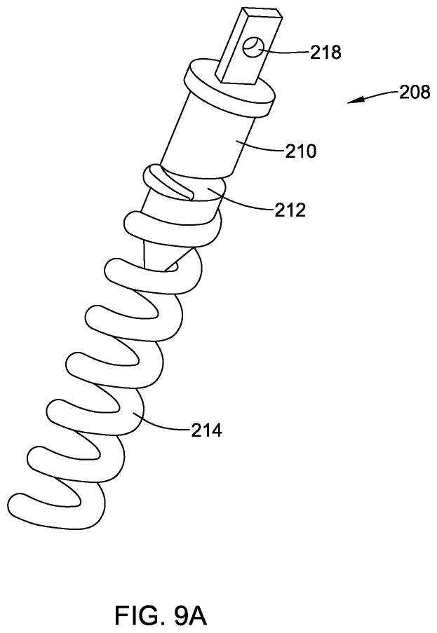

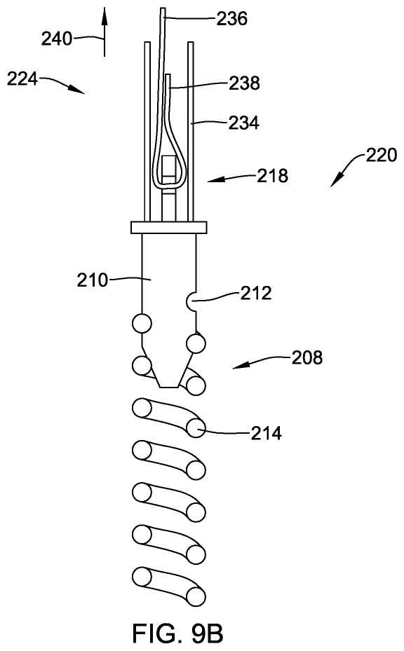

FIG. 9 is a perspective view of an illustrative adjustable endoluminal mitral valve ring implant 200 that is an example of an implantable medical device that may be implanted as part of the assemblies 10 and 30, for example. The adjustable endoluminal mitral valve ring implant 200 may be implanted in order to constrict or otherwise tighten up a patient's mitral valve. The adjustable endoluminal mitral valve ring implant 200 includes a frame 202 that encircles the adjustable endoluminal mitral valve ring implant 200 and includes a number of frame struts 204, two of which are labeled. A number of sliders 206 are disposed over a portion of adjoining frame struts 204.

While not visible in this view, there is a threaded element underneath the slider 206, that is secured relative to the frame 202. Each threaded element is configured to threadedly engage the slider 206 such that rotation of the threaded element relative to the slider 206 causes the slider 206 to translate relative to the adjoining frame struts 204. Rotating the threaded element in a first direction may cause the slider 206 to move downward (in the illustrated orientation), thereby urging the adjoining frame struts 204 towards each other, thereby reducing an overall diameter of the frame 202. Rotating the threaded element in an opposing second direction may cause the slider 206 to move in an opposite direction, for example.

The adjustable endoluminal mitral valve ring implant 200 includes a number of anchor assemblies 208 that may be threaded into tissue proximate the mitral valve. FIG. 9A is a perspective view of one of the anchor assemblies 208. Each anchor assembly 208 includes a body 210 that may be cylindrical or substantially cylindrical in shape. The body 210 includes a helical groove 212 that is adapted to accommodate a helical coil 214. In some cases, and with reference back to FIG. 9, the frame 202 may include an anchor subassembly 216 that may be part of the frame 202, or may be separately formed and then secured to the frame 202, in order to guide the helical coil 214. Rotation of the anchor assembly 208 may cause the helical coil 214 to rotate and translate relative to the anchor subassembly 216 and to extend into tissue, for example, in order to anchor the adjustable endoluminal mitral valve ring implant 200.

The body 210 includes a delivery aperture 218 that may be used to releasable secure the anchor assembly 208, and hence the adjustable endoluminal mitral valve ring implant 200, to a delivery device. It will be appreciated that the adjustable endoluminal mitral valve ring implant 200 may be delivered in a compressed configuration in which each of the sliders 206 are parallel or at least substantially parallel to each other, thereby providing a minimal diameter for delivery.

FIG. 9B shows an assembly 220 that includes the anchor assembly 208 secured relative to an illustrative delivery device 224. The delivery device 224 includes an attachment sheath 234. A release cord 236 extends down through the attachment sheath 234 and passes through the delivery aperture 232. The release cord 236 includes a distal tip region 238 that is beyond the delivery aperture 232 and is disposed within an interior of the attachment sheath 234. Much like the delivery devices 14 and 34, shown in FIGS. 1A-1E and FIGS. 2A-2E, respectively, having the attachment sheath 234 overlying the distal tip region 238 of the release cord 236 means that the release cord 236 is constrained from being pulled out of the delivery aperture 232. Once the adjustable endoluminal mitral valve ring implant 200 has been delivered, moving the attachment sheath 234 in a direction indicated by an arrow 240, the release cord 236 is no longer constrained, and can be withdrawn proximally to release the adjustable endoluminal mitral valve ring implant 200.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An assembly for delivering an implantable medical device, the assembly comprising:
an implantable medical device including an attachment feature; and
a delivery device adapted to releasably secure the implantable medical device, the delivery device including an attachment element adapted to create an interference fit with the attachment feature of the implantable medical device;
wherein the delivery device is adapted to enable a user to selectively disengage the interference fit between the attachment element of the delivery device and the attachment feature of the implantable medical device with a short linear motion when the delivery device is positioned proximate a delivery site for the implantable medical device.

2. The assembly of claim 1, wherein the interference fit between the attachment element of the delivery device and the attachment feature of the implantable medical device enables the implantable medical device to pivot relative to the delivery device before the interference fit is disengaged by the user.

3. The assembly of claim 1, wherein the delivery device comprises an attachment sheath and the attachment element comprises a release cord adapted to extend through the attachment sheath and engage the attachment feature of the implantable medical device, the release cord including a distal region that is adapted to extend back into the attachment sheath.

4. The assembly of claim 3, wherein the short linear motion comprises moving the attachment sheath a distance of about ten millimeters or less in a proximal direction, thereby freeing the distal region of the release cord from the attachment sheath.

5. The assembly of claim 4, wherein the release cord is adapted to enable the release cord to be retracted proximally into the attachment sheath once the attachment sheath has been moved proximally.

6. The assembly of claim 3, wherein the distal region has an outer diameter that is greater than an outer diameter of a rest of the release cord.

7. The assembly of claim 3, wherein the distal region has an outer diameter that is equal to an outer diameter of a rest of the release cord.

8. The assembly of claim 1, further comprising a moveable element having a first configuration in which the moveable element is adapted to hold the attachment element in the interference fit with the attachment feature and a second configuration in which the moveable element is adapted to release the attachment element from its interference fit with the attachment feature.

9. The assembly of claim 8, wherein:
the one or more attachment features comprises an attachment cavity formed within the implantable medical device;
the one or more attachment elements comprise an attachment member that fits within the attachment cavity; and
the moveable element is adapted to move within the attachment member between a release position in which the attachment member is released from an interference fit with the attachment cavity and a secure position in which the attachment member is held in an interference fit with the interference cavity.

10. The assembly of claim 9, wherein the short linear motion comprises moving the moveable element a distance of about ten millimeters or less in a proximal direction.

11. The assembly of claim 9, wherein the moveable element comprises a pin that is moveable within a lumen extending within the attachment member.

12. The assembly of claim 9, wherein:
the one or more attachment features comprises an attachment cavity formed within the implantable medical device, the attachment cavity including a toroidal spring; and
the one or more attachment elements comprise a rod having an annular groove adapted to accommodate the toroidal spring.

13. An assembly for delivering an implantable medical device, the assembly comprising:
an implantable medical device including an attachment feature; and
a delivery device adapted to releasably secure the implantable medical device, the delivery device comprising:
an attachment sheath, the attachment sheath adapted to be linearly moveable a short distance; and
a release cord adapted to extend through the attachment sheath and releasably engage the attachment feature, the release cord including a distal region that is adapted to extend back into the attachment sheath.

14. The assembly of claim 13, wherein the attachment sheath is adapted to be linearly moveable a short distance of about ten millimeters or less, thereby freeing the distal region of the release cord from the attachment sheath.

15. The assembly of claim 14, wherein the release cord is adapted to enable the release cord to be retracted proximally into the attachment sheath once the attachment sheath has been moved proximally.

16. The assembly of claim 13, wherein the distal region has an outer diameter that is greater than an outer diameter of a rest of the release cord.

17. The assembly of claim 13, wherein the distal region has an outer diameter that is equal to an outer diameter of a rest of the release cord.

18. An assembly for delivering an implantable medical device, the assembly comprising:
an implantable medical device including an attachment cavity formed within the implantable medical device; and
a delivery device adapted to releasably secure the implantable medical device, the delivery device comprising:
an attachment member that is adapted to releasably fit within the attachment cavity, the attachment member including a lumen extending within the attachment member;
a moveable element slidingly disposed within the lumen, the moveable element moveable between a first configuration in which the moveable element is adapted to hold the attachment element in the interference fit with the attachment cavity and a second configuration in which the moveable element is adapted to release the attachment member from its interference fit with the attachment cavity.

19. The assembly of claim 18, wherein the moveable element is adapted to move axially within the lumen a distance of about ten millimeters or less in order to move between the first configuration and the second configuration.

20. The assembly of claim 18, wherein the moveable object comprises a pin.
